(19) United States
(12) Patent Application Publication
(10) Pub. No.: US 2014/0155819 A1
(43) Pub. Date: Jun. 5, 2014

(54) Medicament Delivery Systems

(71) Applicant: PICOLIFE TECHNOLOGIES, Deerfield, IL (US)

(72) Inventors: Farid Amirouche, Highland Park, IL (US); Julien Jonvaux, Wilmette, IL (US)

(73) Assignee: PicoLife Technologies, Deerfield, IL (US)

(21) Appl. No.: 13/692,868

(22) Filed: Dec. 3, 2012

(51) Int. Cl. A61M 5/14; A61M 5/158; A61M 5/172

(52) U.S. Cl. CPC A61M 5/1407; A61M 5/1723; A61M 5/158
USPC 604/82; 604/66; 604/173

(57) ABSTRACT

Embodiments of the disclosure may include a medical device for delivering medicament to a body of a user. The device may include a connector for channeling a fluid from a fluid source and a device platform configured to receive the fluid via the connector. The device platform may contact the user's body. The device may further include one or more fluid cannulae coupled to and extending from the device platform and configured for insertion into the body. The fluid cannulae may be configured to receive the fluid from the device platform. The device may also include one or more channels configured to channel the fluid through the connector, into the device platform, into the one or more fluid cannulae, and into the body. The device may also include one or more sensors coupled to the device platform and configured for monitoring a parameter of the body.

### FIELD OF THE DISCLOSURE

Embodiments of the present disclosure relate to, among other things, medical devices and, in particular, to devices for the delivery of one or more substances, such as, e.g., medicaments, to a user by, for example, topical and/or subcutaneous delivery.

### BACKGROUND OF THE DISCLOSURE

Diabetes is a complex disease caused by the body's failure to produce adequate insulin or a cell's failure to respond to insulin, resulting in high levels of glucose in the blood. Type I diabetes is a form of diabetes that results from autoimmune destruction of insulin-producing beta cells of the pancreas in genetically predisposed individuals. There is currently no cure, and treatment by either the injection or infusion of insulin must be continued indefinitely. Type II diabetes is a metabolic disorder that may be brought on at any age and may be caused by a combination of lifestyle, diet, obesity, and genetic factors. The World Health Organization recently revised its findings from a study conducted in 2004 with predictions that by 2030, 10% of the world's population of all ages will have either Type I or Type II diabetes. This translates to roughly 552 million people worldwide suffering from some form of this disease.

Typically, treatment for diabetes may require both repeated checking of blood glucose levels and several injections of insulin throughout the day, as prescribed by a physician, because insulin cannot be taken orally. Major drawbacks of such treatment may include the need to draw blood and test glucose levels throughout the day, improper or low dosage amounts of insulin, contamination of the insulin delivery system, lifestyle restrictions, the potential development of subcutaneous scar tissue due to repeated injections at the same location, and the high cost of medication, testing strips, and other treatment-related materials.

Diabetes may be controlled by insulin replacement therapy, in which insulin is delivered to the diabetic person by injection to counteract elevated blood glucose levels. Therapies may include the basal/bolus method of treatment in which a basal dose of a long-acting insulin medication, such as, for example, Humalog® or Apidra®, is delivered via injection once every day, or, in the alternative, gradually throughout the day. The basal dose may provide the body with an insulin profile that is relatively constant throughout the day, or could follow a profile tailored for the particular diabetic patient. These rates may change based on the patient's response to insulin. At mealtime, an additional dose of insulin, or a bolus dose, may be administered based on the amount of carbohydrate and protein in the meal. The bolus dose may be viewed as an emergency response to spikes in blood sugar that may be brought down or otherwise controlled by injection of insulin. Accurate calculations of various parameters, including, but not limited to, the amount of carbohydrates and proteins consumed, and the lapse in time since the last dosage, may be necessary to determine the appropriate dosage of insulin. The dosages are thus prone to human error, and the method may be ineffective when doses are skipped, forgotten, or miscalculated. Exercise, stress, and other factors can also cause the calculations to be inaccurate. Bolus doses are usually administered when the patient's glucose level is high or above certain acceptable thresholds and needs immediate attention.

To address these and other problems, insulin delivery devices were developed to mimic the way a normal, healthy pancreas delivers insulin to the body. Innovations strove to improve diabetic treatment by, for example, increasing patient compliance with treatment and helping to decrease the number of hyper- and hypoglycemic events. Ambulatory devices often focused on improving portability and discreteness, but these bolus delivery systems have various drawbacks. Additionally, while disposable insulin devices have increased in popularity, the cost to the patient of such devices has also increased approximately 62% per year.

One problem with many miniaturized infusion devices is that they may need to be carried or remain around the injection site at all times, which may be inconvenient when travelling or during certain activities, for example. Further, some devices may not allow for the reusability of a needle for multiple insertions. This means that infusion devices, such as infusion sets, may need to be replaced every few days, whereby the connection to the insulin pump may need to be disconnected to the new injection location. This may result in the complete replacement of the infusion set located near the injection point.

Additionally, the devices may be bulky and may be visible even when located underneath a patient's clothing. Some infusion devices have been designed to incorporate a lower profile, thus reducing the device height when adjacent the body of a user. However, these devices may require cannula or needles to be inserted into a user at a shallower angle, thus resulting in an increased cannula or needle length to reach a similar depth of insertion compared to standard infusion devices. This extension of the cannula or needle may increase the overall footprint of the device on the skin.

Another recurring problem with many miniaturized ambulatory infusion devices is that the number of medications that can be delivered via the devices often cannot meet the needs of certain diabetic patients. Many diabetics may require multiple types of medication. Some devices have been developed that use multiple cannulae for the delivery of multiple medicament types. However, this approach may require more rigid control of fluid deliver rates flowing through the separate injection catheters to ensure the resulting medicament mixture is properly mixed. No provisions may be provided if differing amounts of fluid medicaments are required. Therefore, a substantial need exists to increase the number of medications that can be efficiently delivered to address the needs of both Type I and Type II diabetic patients.

Diabetes patients may need to take repeated glucose readings to help track glucose trend information. Available infusion sets may not contain a continuous glucose monitoring system. Instead, patients may need to attach a separate, continuous glucose monitoring platform to their insulin pump, resulting in a potentially cumbersome system of tubing and wires. Further, attempts at incorporating patient monitoring into infusion sets may require sensor electrodes located close to the infusion site. This orientation may disturb the signals that the sensor element receives, giving a false reading of the measured characteristic. Thus, a need exists for an infusion set with a built-in, continuous glucose monitoring system that may streamline testing and drug delivery into one component and may be less noticeable to the patient and observers. An infusion set with a continuous glucose monitoring system may also be more cost effective than buying a separate glucose system.

Embodiments of the disclosure described herein may overcome some disadvantages of the prior art.

### SUMMARY OF THE DISCLOSURE

Embodiments of the present disclosure relate to medical devices, such as devices for releasing a medicament to the body of a wearer. Various embodiments of the disclosure may include one or more of the following aspects.

In accordance with one embodiment, a medical device for delivering medicament to a body of a user may include a connector for channeling a fluid from a fluid source and a device platform configured to receive the fluid via the connector. The device platform may contact the user's body. The device may further include one or more fluid cannulae coupled to and extending from the device platform and configured for insertion into the body. The fluid cannulae may be configured to receive the fluid from the device platform. The device may also include one or more channels configured to channel the fluid through the connector, into the device platform, into the one or more fluid cannulae, and into the body. The device may also include one or more sensors coupled to the device platform and configured for monitoring a parameter of the body.

Various embodiments of the adaptor may include one or more of the following features: the one or more sensors may be included in the sensor cannula coupled to the device platform and the sensor cannula may be configured for insertion into the body of the user; the one or more sensors may be wirelessly coupled to a controller located external to the medical device; the one or more sensors may include a continuous blood glucose monitor and the fluid may include insulin; the one or more channels may include at least one channel configured to carry one or more sensor wires from the one or more sensors, through the device platform, through the connector, and to a proximal region of the medical device; and data received from the one or more sensors may be used to control the delivery of fluid to the user.

In accordance with another embodiment, a medical device for delivering medicament to a body of a user may include a connector for channeling at least a first fluid and a second fluid from one or more fluid sources. The device may also include a device platform configured to receive the first fluid and the second fluid from the connector and to contact the body. The device may further include one or more cannulae coupled to the device platform. At least one of the one or more cannulae may be configured to receive the first fluid and the second fluid from the device platform and may be configured for insertion into the body. The device may include one or more channels configured to channel the first fluid and the second fluid through the connector, into the device platform, into the one or more cannulae, and to the body of the user.

Various embodiments of the medical device may include one or more of the following features: the first fluid and the second fluid may be different types of medicaments; the one or more channels may include a first channel for carrying the first fluid and a second channel for carrying the second fluid so that the first fluid and the second fluid are delivered separately to the body of the user; the one or more channels may include a first channel for carrying the first fluid and a second channel for carrying the second fluid, wherein the first channel and the second channel join together inside of the medical device to form a combined channel, such that the first fluid and the second fluid are carried together in the combined channel; and the device platform may further comprise a mixing chamber configured to mix the first fluid and the second fluid, wherein the first channel and the second channel are configured to channel the first fluid and the second fluid into the mixing chamber, and the combined channel is configured to channel the first fluid and the second fluid into the one or more cannulae for delivery to the body of the user.

In accordance with another embodiment, a medical device for delivering medicament to a body of a user may include a connector for channeling a fluid from a fluid source and a device platform configured to receive the fluid from the connector. The device platform may be configured to attach to the body of the user. The device may also include one or more cannulae coupled to the device platform that receive the fluid from the device platform and are configured for insertion into the user. The device may also include one or more channels configured to channel the fluid through the connector, into the device platform, into the one or more cannulae, and to the body of the user. The device platform may be configured to rotate such that the one or more cannulae can be withdrawn from the body of the user, such that the one or more cannulae rotates with the device platform, and such that the one or more cannulae is configured to be re-inserted into the body of the user in a different location without detaching the device platform from the body of the user.

Various embodiments of the medical device may include one or more of the following features: the device platform may be configured to allow a user to increase or decrease the angle of insertion of the one or more cannulae into the body of the user; the device platform may include a gap through which the one or more cannulae may extend to contact the body of the user; the one or more channels may extend in an arc around a portion of the device platform such that the length of the one or more channels in the device platform corresponds to the full range of rotation at which the one or more cannulae coupled to the device platform can be rotated; the medical device may include a locking mechanism configured to prevent further rotation of the device platform once a desired rotational position has been achieved; the medical device may further comprise a sensor operably coupled to the device platform and configured to measure a parameter of the user; the sensor may be disposable; the sensor may be configured to attach to the body of the user, the device platform may be configured to rotate, and the sensor may be configured to be detached from the body of the user, rotate with the device platform to a new location, and re-attach to the body of the user in the new location, without removing the device platform from the body of the user; the one or more cannulae may include at least one fluid cannulae configured to receive one or more fluids and at least one sensor cannulae configured to contain one or more sensors; at least one of the one or more fluid cannulae and the one or more sensor cannulae may be disposable and configured to be withdrawn from the body of the user and rotated with the device platform, and at least one of the fluid cannulae and the sensor cannulae may be configured to be replaced with a new fluid cannulae or a new sensor cannulae, and re-inserted into the body of the user; the medical device may be configured to deliver a first fluid and a second fluid to the body of the user; the one or more cannulae may include a first fluid cannulae configured to deliver the first fluid and the second fluid to the body of the user; the one or more cannulae may include a sensor cannulae, a first fluid cannulae, and a second fluid cannulae, wherein the first fluid is delivered to the body of the user through the first fluid cannulae, and the second fluid is delivered to the body of the user through the second fluid cannulae; and the sensor cannulae may include a continuous glucose monitor and the first fluid may be insulin.

### BRIEF DESCRIPTION OF THE DRAWINGS

The accompanying drawings illustrate certain embodiments of the present disclosure, and together with the description, serve to explain principles of the present disclosure.

FIG. 1A depicts a perspective view of a medicament delivery device, in accordance with an embodiment of the present disclosure;

FIG. 1B depicts an alternative configuration of the device of FIG. 1;

FIG. 2A depicts a partially exploded view of the device of FIG. 1;

FIG. 2B depicts another partially exploded view of the device of Figure

FIG. 3 depicts an exploded view of a medicament array of the device depicted in FIG. 1;

FIG. 4A depicts a perspective view of an pump outlet of a medicament distribution device, according to an embodiment;

FIG. 4B depicts a perspective view of the opposite side of the pump outlet depicted in FIG. 4A;

FIG. 4C depicts an end view of the pump outlet depicted in FIG. 4A;

FIG. 4D depicts a cut-away view of the pump outlet depicted in FIG. 4A;

FIG. 5A depicts a cut-away view of an inlet interface of a medicament delivery device, in accordance with an embodiment of the present disclosure;

FIG. 5B depicts an optional cut-away view of the inlet interface depicted in FIG. 5A;

FIG. 5C depicts a cut-away view of the pump outlet and inlet interface of a medicament delivery device, in accordance with an embodiment of the present disclosure;

FIG. 5D depicts an optional cut-away view of the pump outlet and inlet interface depicted in FIG. 50;

FIG. 6A depicts a perspective view of an connector of a medicament delivery device, in accordance with an embodiment of the present disclosure;

FIG. 6B depicts a top view of the connector depicted in FIG. 6A;

FIG. 6C depicts a cross-sectional view of a portion of the connector depicted in FIG. 6A;

FIG. 7A depicts a perspective view of a base platform of a medicament delivery device, in accordance with an embodiment of the present disclosure;

FIG. 7B depicts an alternative perspective view of the base platform of FIG. 7A;

FIG. 7C depicts a top view of the base platform of FIG. 7A;

FIG. 8A depicts a top, partial cut-away view of a base platform of a medicament delivery device, in accordance with an embodiment of the present disclosure;

FIG. 8B depicts a magnified, cut-away view of the base platform of FIG. 8A;

FIG. 9A depicts a top exploded view of a portion of a base platform and a rotating platform of a medicament delivery device, in accordance with an embodiment of the present disclosure;

FIG. 9B depicts a bottom exploded view of the base platform and the rotating platform of FIG. 9A;

FIG. 9C depicts a side view of the base platform and the rotating platform of FIG. 9A;

FIG. 9D depicts an alternative side view of the base platform and the rotating platform of FIG. 9A;

FIG. 10A depicts a perspective view of a rotating platform of a medicament delivery device, in accordance with an embodiment of the present disclosure;

FIG. 10B depicts a bottom view of the rotating platform of FIG. 10A;

FIG. 10C depicts a top view of the rotating platform of FIG. 10A;

FIG. 11A depicts a side, cut-away view of a rotating platform and medicament cannula of a medicament delivery device, in accordance with an embodiment of the present disclosure;

FIG. 11B depicts a side, cut-away view of a rotating platform and sensor cannula of a medicament delivery device, in accordance with an embodiment of the present disclosure;

FIG. 11C depicts a side, cut-away view of a base platform, a rotating platform, medicament cannula and medicament cannulae introducer of a medicament delivery device, in accordance with an embodiment of the present disclosure;

FIG. 11D depicts a side, cut-away view of a base platform, a rotating platform, sensor cannula and sensor cannula introducer of a medicament delivery device, in accordance with an embodiment of the present disclosure;

FIG. 12A depicts a perspective view of a medicament cannulae of a medicament delivery device, in accordance with an embodiment of the present disclosure;

FIG. 12B depicts a cut-away view of the medicament cannulae of FIG. 12A;

FIG. 13A depicts a perspective view of a sensor cannula of a medicament delivery device, in accordance with an embodiment of the present disclosure;

FIG. 13B depicts a cut-away view of the sensor cannula of FIG. 13A;

FIG. 14A depicts a perspective view of a medicament delivery device, in accordance with an embodiment of the present disclosure;

FIG. 14B depicts a side view of the medicament delivery device of FIG. 14A;

FIG. 14C depicts a side view of the medicament delivery device of FIG. 14A;

FIG. 15A depicts a perspective view of a connector of a medicament delivery device, in accordance with an embodiment of the present disclosure;

FIG. 15B depicts a top view of the connector depicted in FIG. 15A;

FIG. 16A depicts a cut-away view of a medicament delivery device, in accordance with an embodiment of the present disclosure;

FIG. 16B depicts a perspective view of a connector portion of the medicament delivery device of FIG. 16A;

FIG. 17 depicts a perspective view of a medicament delivery device, in accordance with an embodiment of the present disclosure;

FIG. 18A depicts a perspective, cut-away view of a base platform of a medicament delivery device, in accordance with an embodiment of the present disclosure;

FIG. 18B depicts a top, cut-away view of the base platform of FIG. 18A;

FIG. 19A depicts a partial cut-away view of a medicament delivery device, in accordance with an embodiment of the present disclosure;

FIG. 19B depicts a perspective view of the medicament delivery device of FIG. 19A;

FIG. 19C depicts a top, partial cut-away view of the medicament delivery device of FIG. 19A; and

FIG. 19D depicts a cross-sectional view of a portion of the medicament delivery device depicted in FIG. 19A.

### DETAILED DESCRIPTION OF EMBODIMENTS

Reference will now be made in detail to the embodiments of the present disclosure described below and illustrated in the accompanying drawings. Wherever possible, the same reference numbers will be used throughout the drawings to refer to same or like parts.

While the present disclosure is described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the invention. Accordingly, the disclosure is not to be considered as limited by the foregoing or following descriptions.

Other features and advantages and potential uses of the present disclosure will become apparent to someone skilled in the art from the following description of the disclosure, which refers to the accompanying drawings.

Prior to providing a detailed description of the embodiments disclosed herein, however, the following overview is provided to generally describe the contemplated embodiments. Further, although the embodiments disclosed herein are described in connection with infusion sets capable of monitoring blood glucose, those of ordinary skill in the art will understand that the principles of the present disclosure may be suitable for monitoring any body parameter, including, e.g., blood pressure, cholesterol levels, sodium levels, medicament saturation levels, and so forth. Further, although the embodiments disclosed herein are described in connection with delivery of, e.g., insulin to treat diabetes, those of ordinary skill in the art will understand that any suitable therapeutic agent may be delivered to a user, regardless of whether the agent is delivered to treat a disease state. For example, the embodiments disclosed herein may deliver medicaments for pain management or joint lubrication, or may be used for reverse controlled fluid extraction.

The disclosed embodiments relate to a miniature medicament delivery device, such as an integrated, continuous, glucose monitoring device. The term “fluid” may include a state of matter or substance (liquid, gas, or a mixture of liquid and gas), whose particles can move about freely and have no fixed shape, but rather conform to the shape of their containers. The term “channel” may include a passage for fluids to flow through. Further, the term “medicament” may be used to refer to a substance used in therapy, a substance that treats, prevents, or alleviates the symptoms of disease, a medicine in a specified formulation, an agent that promotes recovery from injury or ailment, or any other fluid used in the treatment or diagnosis of a patient. Commercial applications may include, e.g., home care, hospitals, nursing homes, military, and the battlefield.

Embodiments of the disclosure described herein may overcome at least certain disadvantages of the prior art and may provide a medicament delivery infusion device that may include one or more cannulae, a device platform base with a patch, a pump connector, and a continuous glucose monitoring sensor. Cannula as used herein may include any suitable flexible or rigid catheter, needle, or tube configured to carry fluid. The glucose monitoring sensor, such as a transcutaneous continuous monitoring system, may be integrated as part of the medicament delivery device. The continuous glucose monitoring sensor may, for example, be included as part of a continuous glucose monitoring cannula, which may be configured for insertion into a user, e.g., subcutaneously. Embodiments of the present disclosure may rely on data obtained from the continuous glucose monitoring sensor, which may be fully or partially imbedded into the user's body, and/or in conjunction with a manual test strip reader, to determine the basal and bolus insulin levels to be delivered to the user. Data from the sensor may also be used to determine the type, amount, or rate, e.g., of medicament to be delivered to the user. Exemplary continuous glucose metering and monitoring sensor devices are described in U.S. patent application Ser. No. 13/448,013, filed on Apr. 16, 2012, and U.S. patent application Ser. No. 13/470,140, filed on May 11, 2012, both of which are incorporated in their entirety herein by reference.

In some instances, embodiments of the present disclosure may also be configured to receive data that is obtained by a separate sensing device and then automatically or manually entered into the medicament delivery device, or any associated component thereof. In some embodiments, multiple different types of sensors may be incorporated into the device and different data from the different sensors may be used to track or control user wellness and delivery regimens. This data may become part of an algorithm that automatically delivers the desired bolus amount of medication into the user's body. In some embodiments, the device may calculate the user's blood glucose level and/or other patient parameters, and the result may be displayed on the display of the device. In addition, any suitable means of communicating the user's blood glucose level may be employed. Such means may include, but is not limited to, e.g., an audible announcement of calculated glucose level, a vibratory indication, and/or a tactile indication.

Further, embodiments of the present disclosure may include a rotating platform on the device that may allow the user to adjust the infusion site location. In some embodiments, the rotating portion may be integrated into the device base, and in other embodiments, it may be a separate element rotatably coupled to the device base. Additionally, embodiments of the present disclosure may be configured to allow adjustment of the angle of insertion at the infusion site location. For example, a portion of base platform 400 or rotating platform 500 may raise or lower, device 100 may tilt, or cannulae 600, 700 may be retracted or extended to allow a user to adjust, e.g., increase or decrease, the angle of insertion of cannulae 600, 700.

Some physicians may prescribe additional drugs to their patients, along with insulin, to help manage diabetes. Embodiments of the present disclosure may allow for the delivery of more than one drug at a time by providing a platform that accommodates multi-medicament delivery. This may be accomplished in a number of ways. For example, drugs may be delivered separately, either simultaneously or at controlled sequences, through separate cannulae in the device. In some embodiments, the device may include a mixing chamber that allows multiple medications, e.g., insulin and another medication, for example, glucagon, to be mixed and then delivered together through a single cannula. In another embodiment, a pump may pre-mix the medicaments before delivering the drugs to the infusion set device. The device may allow different medicaments to be delivered to the user in different manners, e.g., at different rates, times, pressures, or quantities.

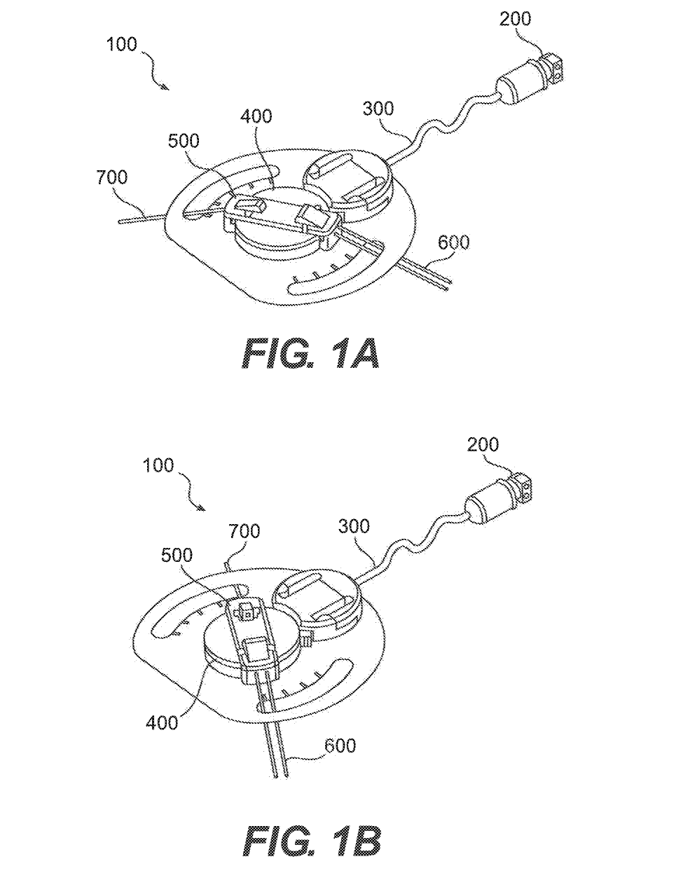

Referring now to the drawings, FIGS. 1A and 1B illustrate a medicament delivery device 100, in accordance with an embodiment of the present disclosure. Device 100 may be configured for delivering multiple medicaments, e.g., two or more medicaments, and may include a continuous glucose monitoring system. In the embodiment depicted in FIGS. 1A and 1B, device 100 may include a pump outlet 200, a connector 300, a base platform 400, a rotating platform 500, two medicament delivery cannulae 600, and a sensor cannula 700. Connector 300 may include two main connection regions: one region that connects to pump outlet 200 and one region that connects to base platform 400. Cannulae 600 may be configured for delivering medicament transcutaneously to the wearer. Cannula 700 may be a continuous glucose monitoring cannula.

Cannulae 600, 700 may be configured to penetrate a user's skin for placement within a user's blood stream for extended periods of time. Cannulae 600, 700 may be rigid or flexible and may be formed of any suitable material. Cannulae 600, 700 may also include any suitable coating desired. For example, the cannula may be coated with anticoagulation, hypoallergenic, anti-inflammatory, and/or antibiotic agents.

Further, any suitable number of cannulae may be included in device 100. Any number of medicament delivery cannulae 600 may be used, for example, one, two, three, or more cannulae. Each cannulae 600 may deliver one or more types of fluid, and may include one or more channels for carrying fluid. Any suitable number and type of sensor cannulae 700 may be included. In some embodiments, one sensor cannula 700 may include multiple sensors for detecting multiple different patient parameters.

Device 100 may be configured to be worn close to or directly in contact with the skin. Device 100 may be worn and completely hidden from view, or may be fully or partially visible. The disclosed device may have any suitable configuration desired. For example, as shown in FIGS. 1A and 1B, device 100 may have an elongated, substantially cylindrical configuration that does not include any sharp edges, thereby allowing the system to be worn or carried by a user, for example, hidden discreetly in, beneath, or attached to a user's clothing (e.g., a shirt or pants). Device 100 may also be available in a variety of sizes. This may allow a user to select a device 100 according to the size or number of dosages or medicaments needed, the length of time over which the user may plan to use device 100, the region of the body on which device 100 may be worn, or the activity level of the user, for example. Further, to facilitate practical, everyday use, device 100 may be lightweight and/or waterproof. Regardless of the specific shape or configuration chosen, the system of the present disclosure may have a substantially low-profile (e.g., slim profile) configuration. That is, the width or overall bulk of the disclosed device 100 may be selected to allow a user to wear the device 100 close to the skin and discreetly in, on, or beneath clothing. This may increase a user's clothing options and/or decrease the psychological effects of wearing the device.

Device 100 may include different colors, designs, or shapes to accommodate the user's preference. Device 100 may also come in any size, depending on the size and/or location of the area to which medicament must be delivered. In some embodiments, device 100 may have designs, colors, shapes, or logos to appeal to children or adults. For example, device 100 may come in a number of shades so as to be inconspicuous and allow for closely matching a wearer's skin color. In other embodiments, device 100 may include images, such as characters, patterns, writing, or team logos, for example.

As shown in FIGS. 1A and 1B, device 100 may include a rotating platform 500 that may be capable of rotating around base platform 400, such that rotating platform 500 and cannulae 600, 700 can be positioned at a variety of angles relative to base platform 400. FIG. 1A depicts rotating platform 500 in a first position, and FIG. 1B shows rotating platform 500 in a second position (rotated approximately 60° with respect to the first position). Any suitable intermediate position of the rotating platform 500 may be possible. For example, in one embodiment, rotating platform 500 may rotate smoothly and may be capable of positioning at any angle. In another embodiment, rotating platform 500 may rotate at set intervals so that platform 500 may be rotated at set increments. These increments may or may not be evenly spaced, and may be permanent or adjustable. Rotating platform 500 may include a dial to allow a user to adjust the angle of rotation. Although FIGS. 1A and 1B show cannulae 600, 700 positioned opposite each other, cannulae 600, 700 may be positioned at any angle relative to each other. Additionally, in some embodiments, the various cannulae may be configured to allow for independent rotation. For example, in some embodiments cannulae 600 and cannula 700 may be rotated separately from each other, such that the angle between them may vary.

Rotating platform 500 may allow a user to change the delivery location of medicament to the wearer. The delivery location may need to be changed, for example, based on the type of medicament to be delivered, the stage of medicament delivery, the treatment stage of the wearer, the comfort of the patient, and the type of drug delivery system that device 100 is connected to. Rotating the delivery location may decrease irritation, pain, inflammation, or scarring for the user. Once the cannulae have been rotated and the insertion location has been changed, device 100 may also include a locking mechanism that may prevent further rotation of rotating platform 500 once a desirable position has been achieved. Additionally, device 100 may include a location marker to allow a user to indicate which injection site locations the user has rotated to, both currently and previously. Any suitable marker may be included, for example, removable tabs or depressible formations.

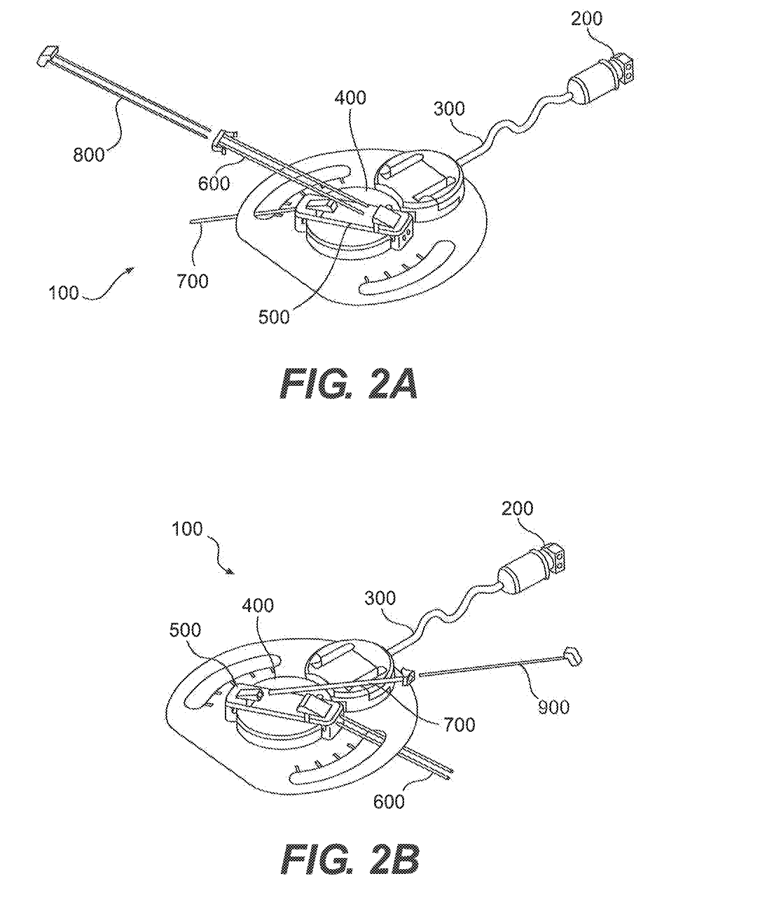

By rotating platform 500, the wearer may be able to adjust the medicament delivery location without fully removing device 100 from his or her body. In some embodiments, a wearer may be able to retain the entire device 100 on the body, removing or retracting only medicament delivery cannulae 600 and sensor cannula 700. In such an embodiment, the wearer may remove or retract the used cannulae 600 and 700 from the skin, as shown in FIGS. 2A and 2B, rotate rotating platform 500 to a new position, and insert either the used cannulae 600, 700 or new cannulae 600, 700 under the skin at the new location. For example, in some embodiments, cannulae 600 or 700 may be disposable and may be configured to be detached from device 100 and be replaced while a portion of device 100 remains attached to the body of the user. In such embodiments, the user may retract used cannulae 600, 700 from the body of the user, remove used cannulae 600, 700 from device 100, attach new cannulae 600, 700 to device 100, rotate rotating platform 500 to a new position, and insert new cannulae 600, 700 in a new location in the body. Rotation of platform 500 may occur before, during, or after replacement of the disposable cannulae. Further, cannulae 600, 700 may also be replaced without rotation of rotating platform 500. In such embodiments, cannulae 600, 700 may be retracted, replaced, and re-inserted into the same location. In some embodiments, the angle of insertion of cannulae 600, 700 may also be changed during the rotation process, or cannulae 600, 700 may be retracted from the body only to change the angle of insertion and without rotation of rotating platform 500. Cannulae introducers 800, 900, shown in FIGS. 2A and 2B, may aid in this process, as described further below.

Alternatively, in still other embodiments, base platform 400 may not be configured to allow for rotation, and device 100 may be stationary. In other embodiments, base platform 400 may itself be rotatable, and device 100 may not include a separate rotating platform 500. Further, device 100 may not include sensor cannula 700, and device 100 may work similarly to what is described above, only without provisions for the removal, rotation, or replacement of sensor cannula 700. In some embodiments, device 100 may include a sensor in non-cannula form, which may be attached to the body, removed, replaced, and rotated in a similar manner. In such embodiments, the non-cannula sensor may or may not be configured for insertion into the patient, for example, a sensor may be configured to rest adjacent to the body of the user or only a portion of the sensor may protrude into the body of the user. The sensor may then be removed from the body of the user, rotated, replaced if disposable, and re-attached to the body of the user without removal of other components of device 100 from the body of the user.

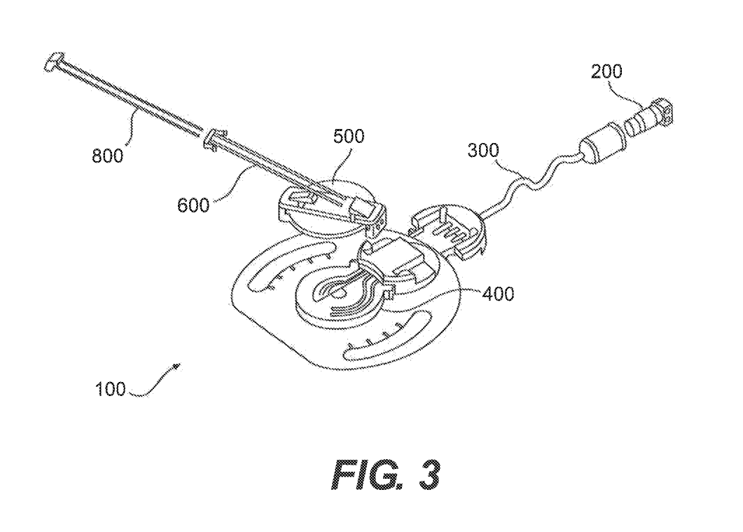

FIG. 3 depicts an exploded view of device 100, without sensor cannula 700, showing the relative positioning of the various parts. FIG. 3 may act as a reference as the individual components are described in detail more fully below. In the embodiment described below, device 100 contains two separate paths for two fluid medicaments. One of skill in the art will appreciate, however, that any suitable number of paths for any suitable number of fluids may be used with device 100.

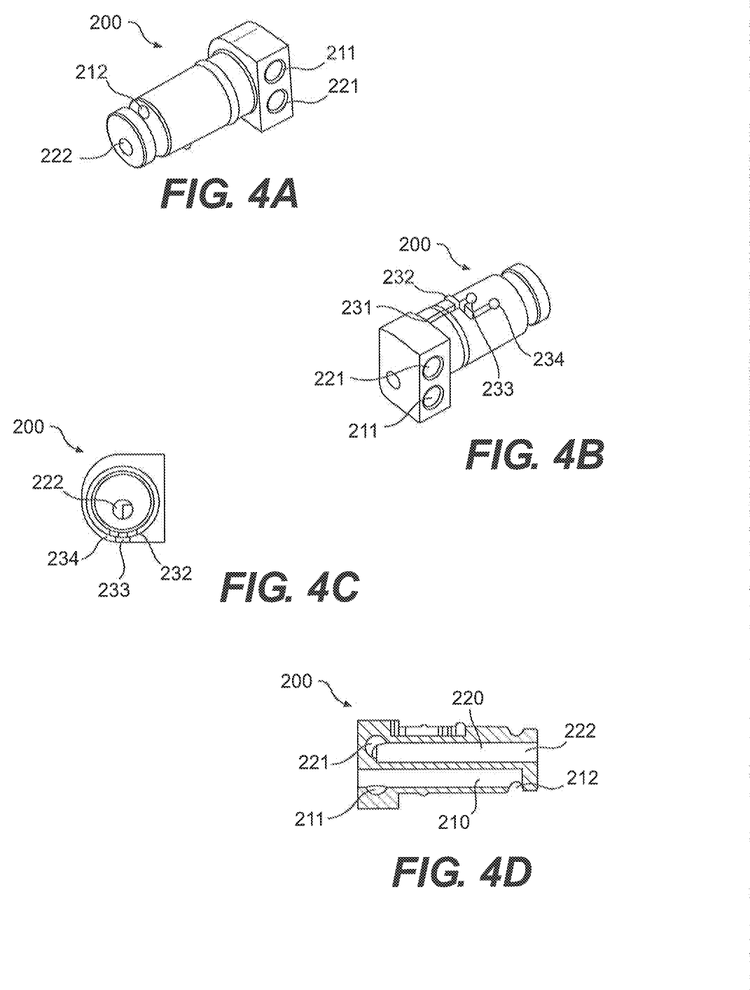

A pump outlet 200 may be located at a region of device 100 located proximal to a medicament source. Pump outlet 200, depicted in FIGS. 4A-4D, may include a first channel 210 that leads a first fluid from a proximal region 211 of pump outlet 200 to a distal region 212 of pump outlet 200, and a second channel 220 that leads a second fluid from a proximal region 221 of pump outlet 200 to a distal region 222. The first fluid and the second fluid may be the same fluid, or they may be different fluids. The one or more fluids may include one or more medicaments.

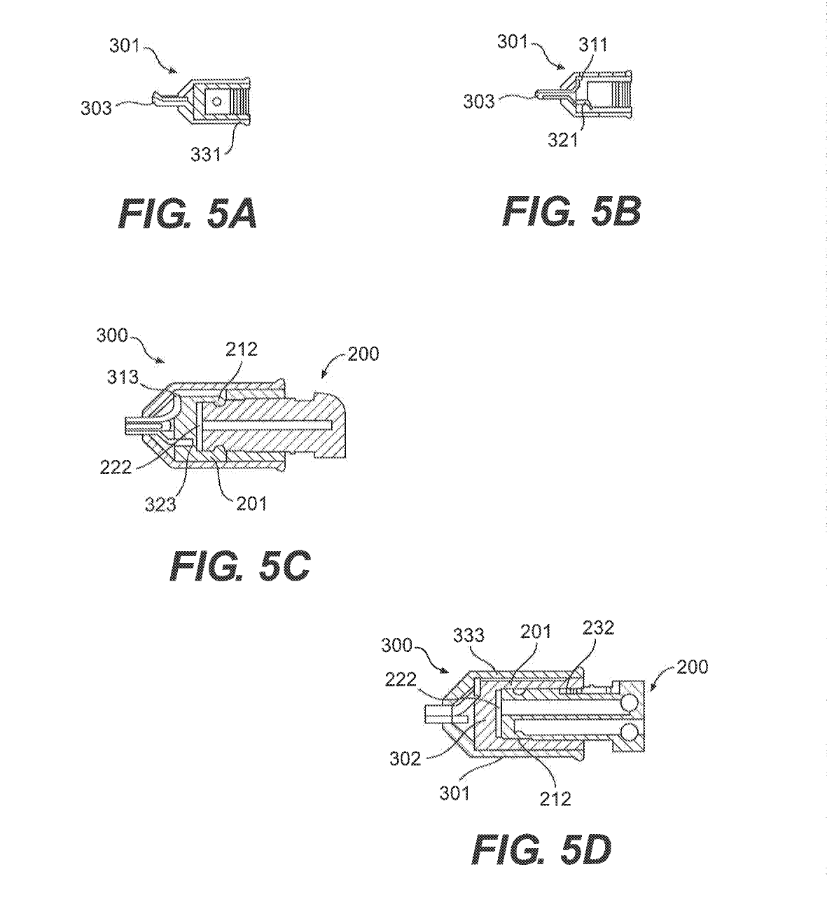

Pump outlet 200 may also include one or more channels 232 configured to contain one or more sensors 233, 234 or the wiring or other components for one or more sensors. Sensors 233, 234 may further include one or more contacts communicating with a channel exit 231 of channel 232 located near a pump. Pump outlet 200 may be configured so as to prevent the first and second fluids from entering channel 232 and contacting sensors 233, 234. For example, as shown in FIGS. 5C and 5D, the interface between pump outlet 200 and connector 300 at location 201 may form a seal that separates fluid channels 210, 220 and electrical contact channel 232. The angled shape of mating surfaces 201 may be similar to those found in Luer taper connections and may form a suitable seal. For example, a 6% taper fitting of mating surfaces 201 may form a seal between distal regions 212, 222 of fluid channels 210, 220, and/or between distal region 212 of fluid channel 210 and electrical contact channel 232. Additionally, a separate seal and/or mating surfaces 201 may be formed of any suitable material, e.g., polypropylene, polyester, or polycarbonate. In some embodiments, one or both of channels 210, 220 may carry insulin, and channel 232 may be configured to carry components, e.g., wires, contacts, and sensors, of a continuous glucose monitor having one or more sensors.

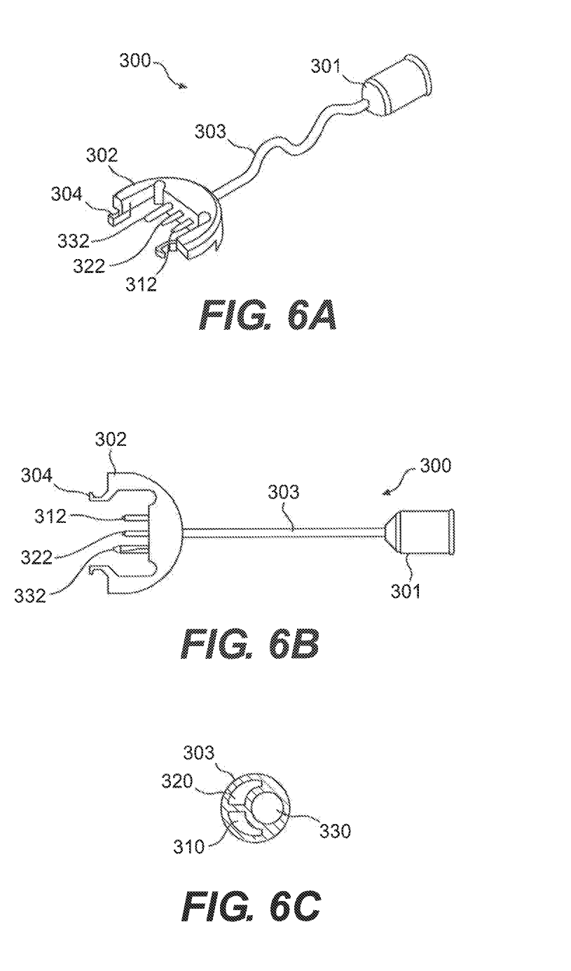

The fluids may exit pump outlet 200 at regions 212, 222 and flow into an inlet interface 301 of connector 300, as shown in FIGS. 5A-5D and FIGS. 6A-6C. Inlet interface 301 may be configured to operably couple to pump outlet 200, such that one or more channels (e.g., one or more of channels 210, 220, and 232) in pump outlet 200 fluidly connect with one or more channels in inlet interface 301 and/or a catheter 303. For example, as is shown in FIGS. 5A-5D, inlet interface 301 may be configured to receive a portion of pump outlet 200. Inlet interface 301 may guide the fluids and sensors (or sensor wires or other components connected to sensors 233, 234) into separate lumens in catheter 303, shown in FIG. 6C. In the depicted embodiment, catheter 303 includes three separate lumens 310, 320, 330.

Catheter 303 may be formed of any suitable tube-like structure for relaying fluids, and may include one or more lumens. Catheter 303 may have any suitable configuration and shape, and may be flexible to permit the relief of stresses imposed on catheter 303 by, e.g., a user's movements. One of skill in the art will appreciate that catheter 303 can be configured to include any number of lumens that may carry any number of fluids and/or electronics, either combined or individually, through these lumens. Further, the lumens in catheter 303 (e.g., one or more of lumens 310, 320, and 330) may align in a 1:1 ratio with channels in pump outlet 200 and/or inlet interface 301, or may include more or less lumens, so that fluids and/or sensor components are combined or divided into the lumens upon exiting pump outlet 200 and catheter 303. Further, in some embodiments, rather than having one catheter 303, connector 300 may include multiple catheters 303.

Lumens 310, 320, 330 may include any suitable shapes and/or configurations. For example, each of lumens 310, 320, 330 may include a substantially circular cross-section configuration. Moreover, the cross-sectional configurations of one or more of lumens 310, 320, 330 may vary relative to the other of lumens 310, 320, 330. Even further, the cross-sectional configuration of one of lumens 310, 320, 330 may vary along its length. In some embodiments, one or more of lumens 310, 320, 330 may be provided with a suitable metering mechanism for controlling the flow of fluids through lumens 310, 320, 330.

The fluids and/or sensor components may enter through inlet interface 301 and exit through base platform interface 302 of connector 300 after passing through lumens 310, 320, 330 of catheter 303. The first fluid may flow from pump outlet 200 through a first pump interface 311, through lumen 310, and into base platform 400 through a first base interface 312. The second fluid may flow from pump outlet 200 through a second pump interface 321, through lumen 320, and into base platform 400 through a second base interface 322. Components for one or more sensors, for example, continuous glucose monitoring sensors, may travel from pump outlet 200 through a third pump interface 331, through lumen 330, and into base platform 400 through a third base interface 332. In another embodiment, the sensor components may originate from a region external from pump outlet 200 and may be introduced into lumen 330 through a different means. The components may be configured to carry signals from the sensors to a proximal region of device 100, or to a region external to device 100. Accordingly, connector 300 may be configured to connect both fluid and electrical components. Alternatively, the sensor may be wireless, and separate channels to hold sensor components may not be needed, as will be discussed further below.

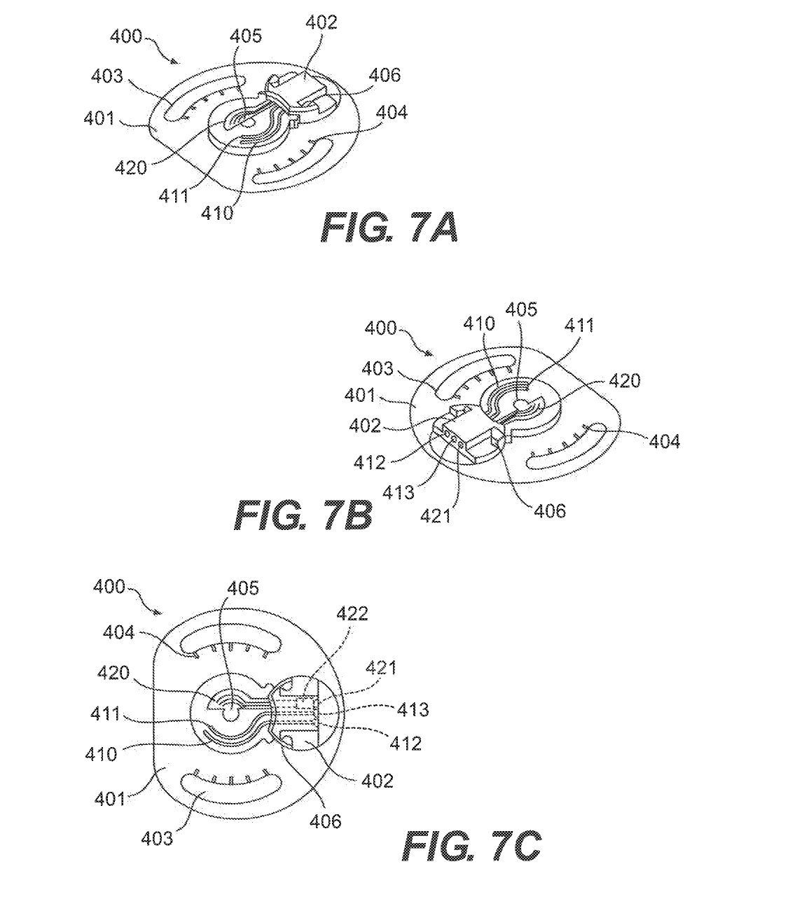

FIGS. 7A-7C show base platform 400, which may serve multiple functions. As will be discussed in further detail, base platform 400 may direct the fluid and sensor components to the correct channels in cannulae 600, 700 and may allow cannulae 600, 700 to be removed and/or changed without removing the entire device 100 from the body of a user. Base platform 400 may also act as the attachment point of device 100 to the user. Base platform 400 may include a patch 401, which may anchor connector 300, base platform 400, and cannulae 600, 700 to the user. Patch 401 may be configured to directly contact the body of a user, e.g., on the skin. Patch 401 may be configured to retain device 100 on the wearer for any amount of time, for example, temporarily or for prolonged use. Patch 401 may be held onto the wearer via any suitable means, for example, with adhesive, such as glue or tape; a strap to wrap around a portion of the body, for example, an elastic, Velcro, hook-and-eye, snap-on, or lace-up strap; suction; or any combination thereof for example. Further, in some embodiments, patch 401 may include a larger band, glove, sock, or other wearable configuration, to wrap around an arm or leg, for example. Such an embodiment may be preferable to aid in stability if distributing medicament to a larger area of the body or for delivering medicament for a shorter time duration.

Patch 401 and components of device 100 may be formed of any suitable materials. Patch 401 may resemble an adhesive bandage, a medical patch, or any other skin-covering device, and may be formed of a suitable plastic or polymer, such as, e.g., silicone, acrylic, rubber, spandex, or natural or synthetic fiber, or any combination of materials. Patch 401 may be, e.g., hypoallergenic, designed to minimize the pulling of body hair, biocompatible, permeable to air, or impermeable to air, depending on the type of medicament to be delivered by patch 401 or the use of device 100. In addition, patch 401 may be waterproof. In some embodiments, patch 401 may be flexible to allow device 100 to move with the user. In other embodiments, patch 401 may be hard, rigid, or inflexible to offer protection to the underlying area. Patch 401 may have multiple layers to allow for the adjustment of rigidity. While patch 401 is depicted as generally circular, patch 401 may be any suitable shape or size and may be configured to attach to any region of the body. For example, patch 401 may be rectangular, oval, or irregularly shaped, and may be small enough to lie flat against a smaller body region, such as a foot or wrist, or large enough to fit over a larger region, such as the abdomen or back.

Further, connector 300, base platform 400, and rotating platform 500 may be made from any suitable materials. Such materials may include, but are not limited to, polymers, plastics, thermoplastics, and/or elastomers. One suitable material may be acrylonitrile butadiene styrene (ABS) or equivalents. Further, the components of device 100 may be made in any suitable size and through any suitable manufacturing process. To facilitate close proximity with the body, device 100 may be formed and/or manufactured with biocompatible materials. The methods used in the manufacture of the polymer components, as well as the arrangement and design of device 100, may be adapted to commonly used sterilization techniques, such as, e.g., gamma irradiation, steam sterilization, or fluid chemical sterilization.

Connector 300, base platform 400, and rotating platform 500 may be provided with any suitable coating desired. For example, one or each of them may be coated with, e.g., hypoallergenic agents to reduce discomfort to a user's skin. Further, they may be provided with a fragrant coating that may please or soothe a user. Instead of a coating, such agents may be impregnated within one or more external walls of connector 300, base platform 400, and rotating platform 500. In addition, base platform 400 may be provided with one or more storage locations to store, e.g., blood glucose test strips. Such storage locations may be secured or unsecured. Moreover, connector 300, base platform 400, and rotating platform 500 may be capable of illumination to indicate use or dispensing of medicaments contained therein. In some embodiments, connector 300, base platform 400, and rotating platform 500 may be configured so that a user can see the medicament being delivered. For example, connector 300, base platform 400, and rotating platform 500 may be formed of a clear or opaque material.

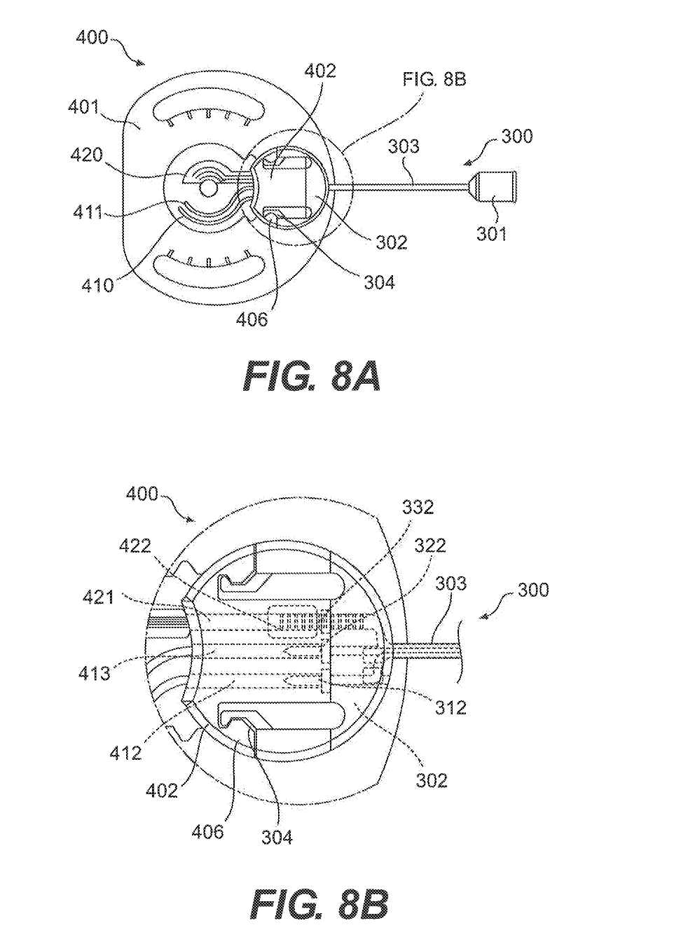

The fluids and sensor components may pass through a catheter interface 402 on base platform 400. As is shown in FIGS. 7A-8B, channels 312, 322, and 332 of connector 300 may fluidly connect to channels 412, 413, and 421, respectively, of base platform 400. Channels 412, 413, and 421 may pass along base platform 400 via channels 410, 411, and 420, respectively, as seen in FIGS. 7A-8B. Connector 300 and base platform 400 may join together by any suitable anchoring mechanism, such as, e.g., clip 304, depicted in FIGS. 6A and 6B. Clip 304 may engage with a mating piece 406 on base platform 400. In other embodiments, connector 300 and base platform 400 may connect via any suitable means, e.g., via a screw fit, a friction fit, a snap fit, a latch mechanism, or by aligning male and female portions, for example.

The connection points between connector 300 and base platform 400 may include any suitable alignment means for aligning the corresponding channels in each portion. For example, one or more alignment holes and projections may be included to help align the channels properly. An alignment hole or projection on connector 300 may align with a corresponding alignment hole or projection on base platform 400. One of ordinary skill in the art will appreciate that the location of alignment holes and projections may be reversible.

Connector 300 may have one standard size, or different sizes may be available to correspond to the size of a user, for example, a child or an adult. In another embodiment, connector 300 may be configured so as to have an adjustable size, e.g., an adjustable length. In such an embodiment, connector 300 may include a release mechanism, e.g. a button, lever, wind, etc., that allows the user to pull more slack out of inlet interface 301 and/or base platform interface 302, for example, so as to lengthen connector 300, or alternatively, to allow extra slack to retract to shorten connector 300. Connector 300, and/or base platform 400 may include a mechanism to lock the length of connector 300 in place once a desired length has been achieved. In such embodiments, the length of the connection between pump outlet 200 and base platform 400 may be adjustable.

Channel 421, as shown in FIGS. 7A-7C, 8A, and 8B, may contain one or more contacts 422 for the separate components for continuous glucose monitoring. For example, channel 421 may contain three contacts, which may include, e.g., a reference electrode, a working electrode, and a counter electrode for use with a transcutaneous continuous monitoring system. As is shown in FIG. 8B, electrical contacts 422 may be arranged in a linear manner along channel 421. Electrical contacts 422 may be arranged in channel 421 in any suitable arrangement and may be regularly or irregularly spaced within channel 421. The fluids may remain in separate channels 412, 413, which may fluidly connect with and extend along channels 410, 411, respectively. Channels 410, 411, 420 may run in an arc around base platform 400. The ends of the arc-shaped channels may correspond to the maximum range of rotation of cannulae 600, 700 on rotating platform 500. In some embodiments, channels 410, 411, 420 may extend around the circumference of base platform 400, forming a loop or a circle rather than two separate arcs. Additionally, certain channels may extend further than other channels in some embodiments, and the channels may be grouped or positioned in any suitable arrangement.

Rotating platform 500 may rotate around a central pivot point 405. The elongation of channels 410, 411, 420 may allow fluids to flow and signals to be passed at any position that rotating platform 500 can reach. Base platform 400 may include one or more gaps 403 through which cannulae 600, 700 may pass to contact the user, as shown in FIGS. 7A-7C. In other embodiments, cannulae 600, 700 may extend past the edge of base platform 400, in which case base platform 400 may not include any gaps 403. In the embodiment shown in FIGS. 7A-7C, cannulae 600, 700 may extend through arc-shaped gaps 403 in the adhesive patch 401, as shown in FIGS. 1A and 1B. Device 100 may also include position indicators, for example, one or more markings 404 on base platform 400, that may serve as visual guides for positioning rotating platform 500 and inserting cannulae 600, 700, e.g., for selecting new cannulae insertion locations. As discussed above, device 100 may also include one or more location markers to allow a user to indicate which injection site locations the user has already rotated to.

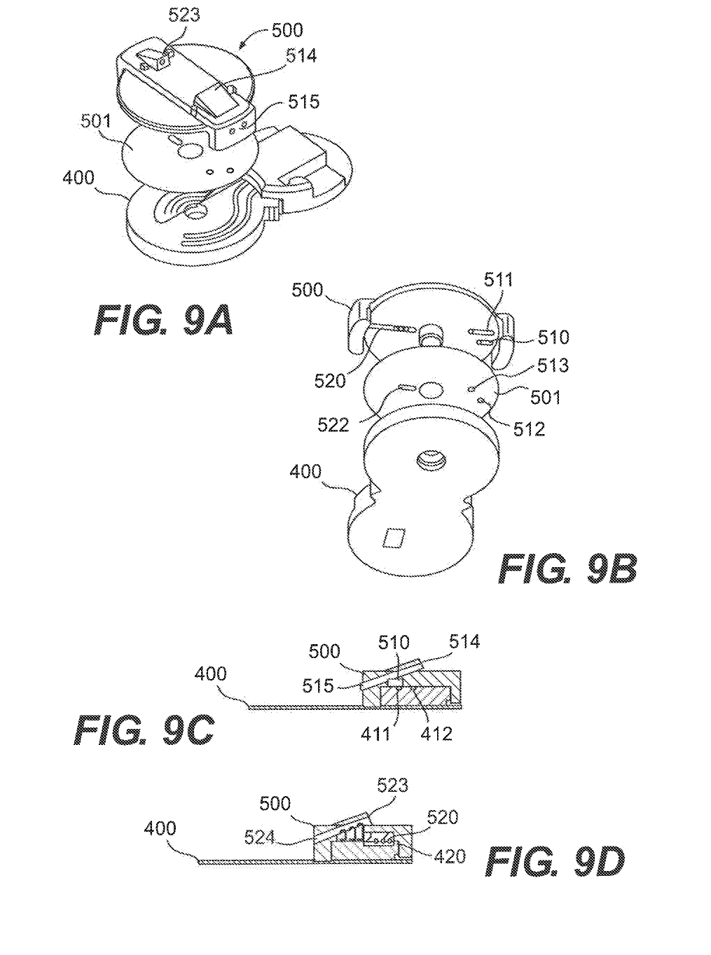

In embodiments in which device 100 includes a rotating platform 500 mounted on base platform 400, rotating platform 500 may engage and rotate relative to base platform 400. As shown in the exploded view of FIGS. 9A-9D, rotating platform 500 may rest on top of and rotate around base platform 400. A membrane 501 may be situated between rotating platform 500 and base platform 400. Membrane 501 may prevent the fluids contained in channels 410, 411 from leaking across channels 410, 411 in base platform 400, or from leaking outside of device 100, or membrane 501 may facilitate rotation, for example.

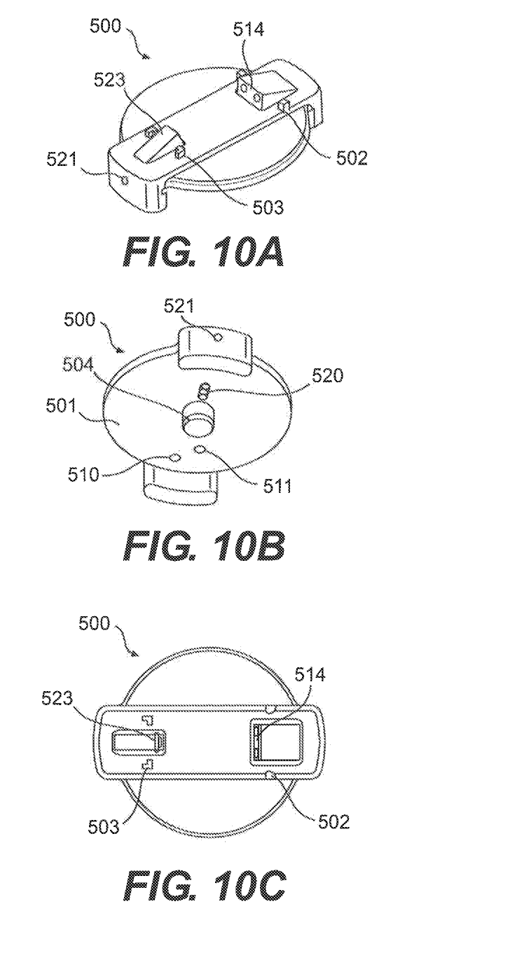

Cannulae 600, 700 may be permanently coupled to device 100 or may be removable from device 100. Cannulae 600, 700 may attach to base platform 400 in any suitable manner. Cannulae 600, 700 may slide into place on rotating platform 500, or may be held by friction fit, snap fit, or any other suitable anchoring mechanism. For example, as shown in FIGS. 9A-9D and 10A-10C, rotating platform 500 may contain insertion points for cannulae 600, 700 and may also include channels for the fluids and sensor wires or other components. Rotating platform 500 may also be configured for use with cannulae introducers 800, 900, to help position cannulae 600, 700 on a user. Cannulae 600, 700 or rotating platform 500 may include one or more locking mechanisms, such as clips 502, 503, to reduce the shifting of cannulae 600, 700 once in place. For example, cannulae 600, 700 may be held in place by flexible clips 603 that deform when inserted into opposing clips 502 on rotating platform 500. While locking mechanisms 502, 503 are depicted as including clip mechanisms, any suitable mechanism may be used to keep cannulae 600, 700 in place.

If included, membrane 501 may include one or more holes 512, 513 for the fluids to pass through. Holes 512, 513 may align with channels 410, 411, respectively, on base platform 400 and channels 510 and 511 on rotating platform 500, allowing fluid to flow from channels 410 and 411, through membrane holes 512 and 513 and through channels 510 and 511. Cannulae 600 and optional cannulae introducer 800 may pass through openings 514 and 515 on rotating platform 500 and may fluidly connect with channels 510 and 511, as seen in FIGS. 9A-9D, 10A-10C, and 11A-11D. Fluid from channels 410 and 411 may flow into cannulae 600 by passing through channels 510 and 511 and flowing into respective cannula openings 610 in cannulae 600, as is shown in 11A (without cannulae introducer 800) and 11C (with cannulae introducer 800).

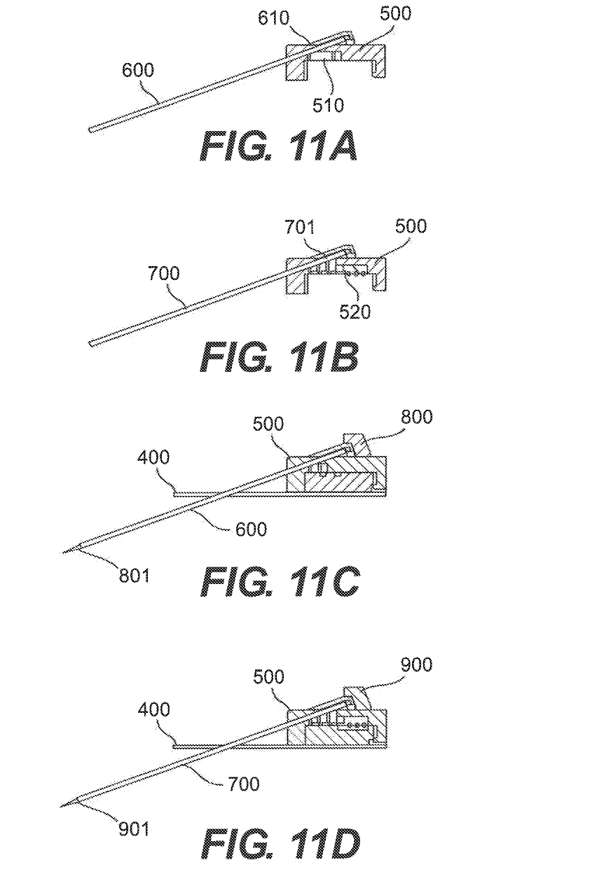

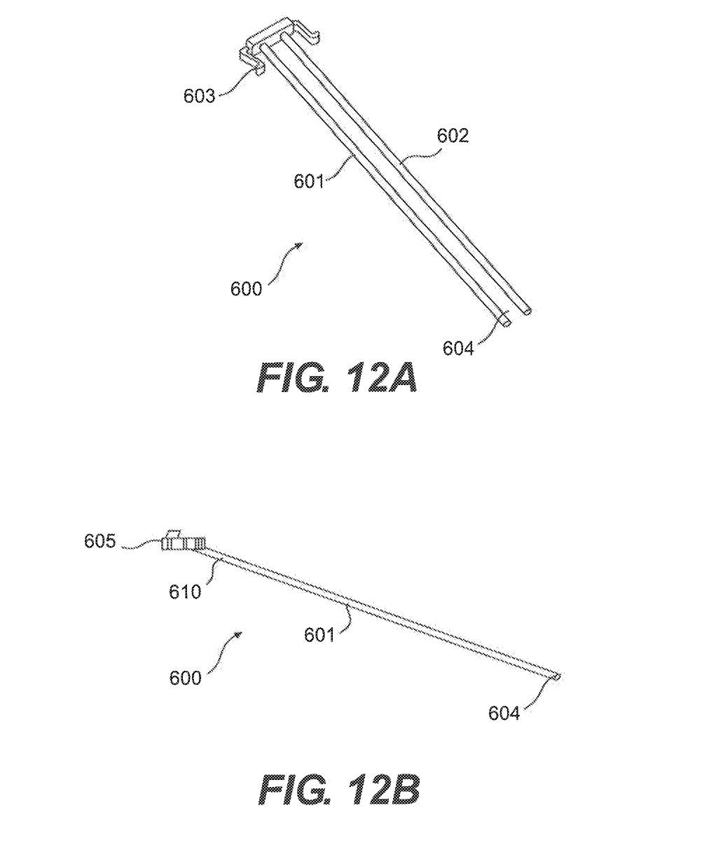

Cannulae 600 may include one or more channels 601, 602 that carry the fluid from rotating platform 500 to a distal end 604 of cannulae 600, as seen in FIGS. 12A and 12B. Fluid may enter from side openings 610 (FIG. 11A) that align with respective channels 510 and 511 in rotating platform 500 and then flow to distal end 604. Cannulae 600 may include seals or membranes 605, e.g., silicone seals or membranes, to prevent the fluids from flowing to unwanted areas. Cannulae 600 may be configured to keep the fluids separate until the fluids have been discharged to the user, for example, injected under the skin of the user.

Membrane 501 may also include one or more holes 522 for the sensor components to pass through. Hole 522 may align with channels 420 on base platform 400 and channel 520 on rotating platform 500, allowing the sensor components to pass through channels 420, through membrane hole 522, and channel 520. Cannula 700 and optional cannula introducer 900 may pass through openings 523, 521 in rotating platform 500 and may fluidly connect with channel 520, as seen in FIGS. 9A-9D, 10A-10C, and 11A-11D. Components from channels 420 may pass through channel 520 and connect with one or more continuous glucose monitoring sensor contacts 701 on sensor cannula 700, as shown in FIGS. 11D (with cannulae introducer 900) and 11D (without cannulae introducer 900).

In the depicted embodiment, rotating platform 500 may rotate both infusion cannulae 600 as well as continuous glucose monitor cannula 700.

Cannulae 600, 700 may be attached to the user, for example, inserted under the skin, with or without the aid of optional cannulae introducers 800, 900, as seen in FIGS. 11A-11D. Introducer tips 801 and 901 may puncture the skin and allow for cannulae 600 and 700, respectively, to be inserted subcutaneously to a desired depth. Cannulae introducers 800, 900 may be removed after insertion has been completed, or in some embodiments, may be configured to remain on device 100. Cannulae 600, 700 may be of any suitable length user. Cannulae 600, 700 may come in standard lengths, or may be customized to suit the user size, activity level, location on the body, or other criteria that may be unique to each user.

Cannulae 600, 700 and introducers 800, 900 may include any suitable tubular structure, including, e.g., catheters, needles, or trocars. Further, cannulae 600, 700 and introducers 800, 900 may be formed of any suitable material. For example, cannulae 600, 700 and introducers 800, 900 may be made of glass, plastic, ceramic, metal (e.g., types of stainless steel, titanium, nitinol), or any suitable combination of materials. For example, the tip portion and the base portion of cannulae 600, 700 and introducers 800, 900 may be comprised of different materials. In some embodiments, the tip portion of cannulae 600, 700 and introducers 800, 900 may be formed of a material that is harder than the base portion. In other embodiments, the entire cannulae or entire introducer may be formed of the same material. Additionally, cannulae 600, 700 and introducers 800, 900 may include any suitable coating, or any suitable combination of coatings. For example, a coating may be lubricious, drug eluting, anticoagulant, antiseptic, anesthetic, etc. Cannulae 600, 700 and introducers 800, 900 may be monolithically formed, or alternatively, may be formed of separate parts made of the same or different materials, for example, plastic-coated glass needles.

In some embodiments, cannulae 600, 700 and introducers 800, 900 may be configured so as to optimize use and/or patient comfort. For example, they may be designed to decrease the amount of force needed to penetrate the skin, or to decrease the amount of force transferred to the skin from the cannulae or optional introducers. In some embodiments, the use of multiple cannulae may decrease the amount of force needed to penetrate the skin.

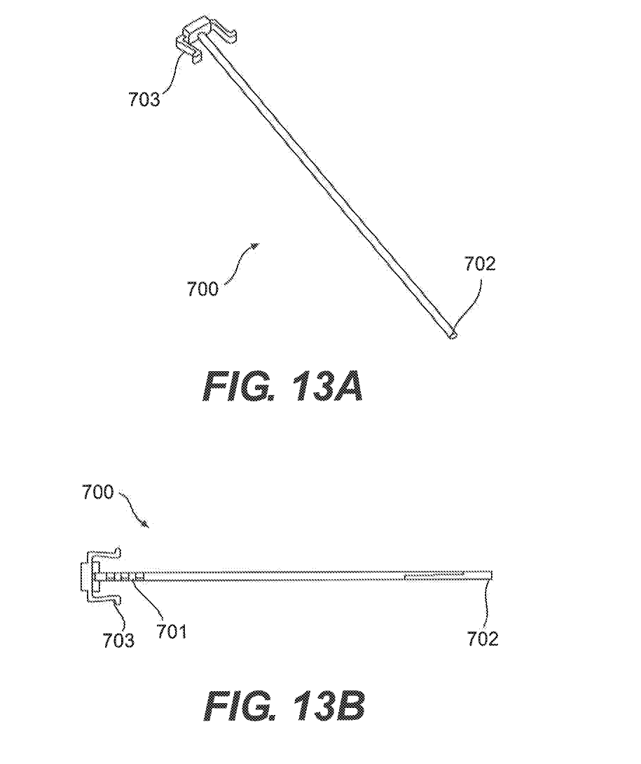

FIGS. 13A and 13B show a continuous glucose monitoring sensor cannula 700. Rotating platform 500 may contact sensor cannula 700 at a cluster of contact points 701 near one extremity of the sensor cannula 700. Contact points 701 are shown arranged in a linear manner in the direction of contact sensor cannula 700, but may be arranged in any suitable manner. Additionally, contact points 701 may be regularly spaced or may be scattered at different distances from one another on device 100. When contact sensor cannula 700 is inserted into rotating platform 500, electrical communication may be facilitated between electrical contacts of rotating platform 500, e.g., lamellar contacts, and contact points 701 of the contact sensor cannula, as shown in FIGS. 9D, 11B and 11D.

Sensor cannula 700 may monitor the blood glucose levels at a tip 702 of sensor cannula 700, which may be located beneath the skin of the user. Sensor cannula 700 may also be held in place by any suitable means, for example, by clips 703 that may deform and latch onto opposing clips 503 on rotating platform 500. Integrating sensor cannula 700 into device 100 may lower the cost of the system and may provide better feedback control and/or more accurate information to the user.

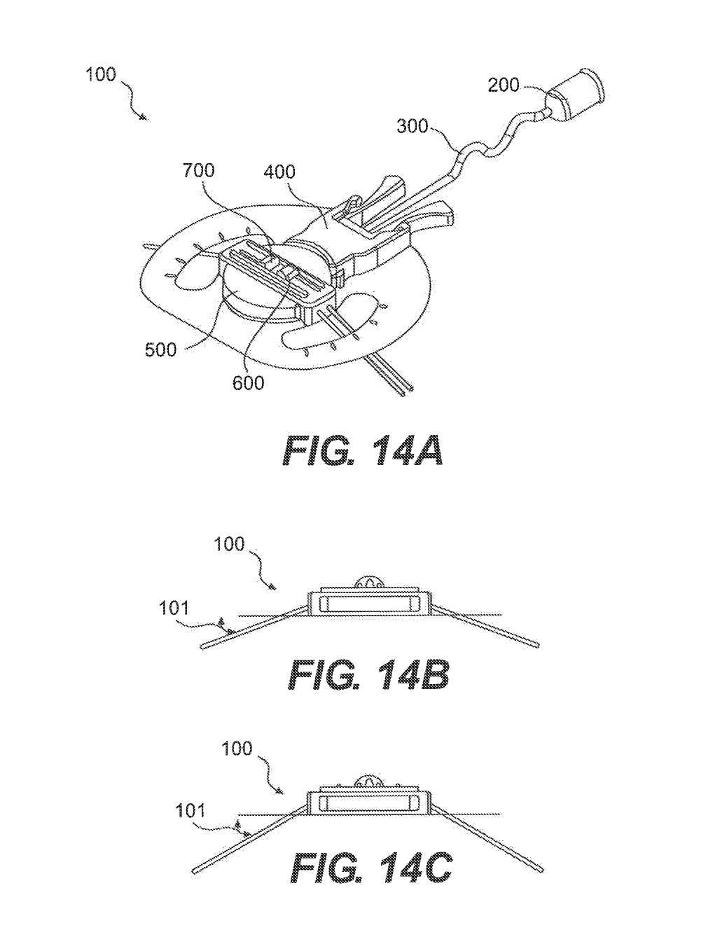

FIGS. 14A-14C, 15A, 15B, 16A, and 16B depict another exemplary embodiment of device 100. As in the previous embodiment, device 100 may include a pump outlet 200, a connector 300, a base platform 400, a rotating platform 500, two medicament delivery cannulae 600, and a sensor cannula 700. Cannulae 600 may be configured to deliver one or more than one fluid, e.g., a fluid medicament, to the body of a user. Cannula 700 may include a continuous glucose monitoring cannula.

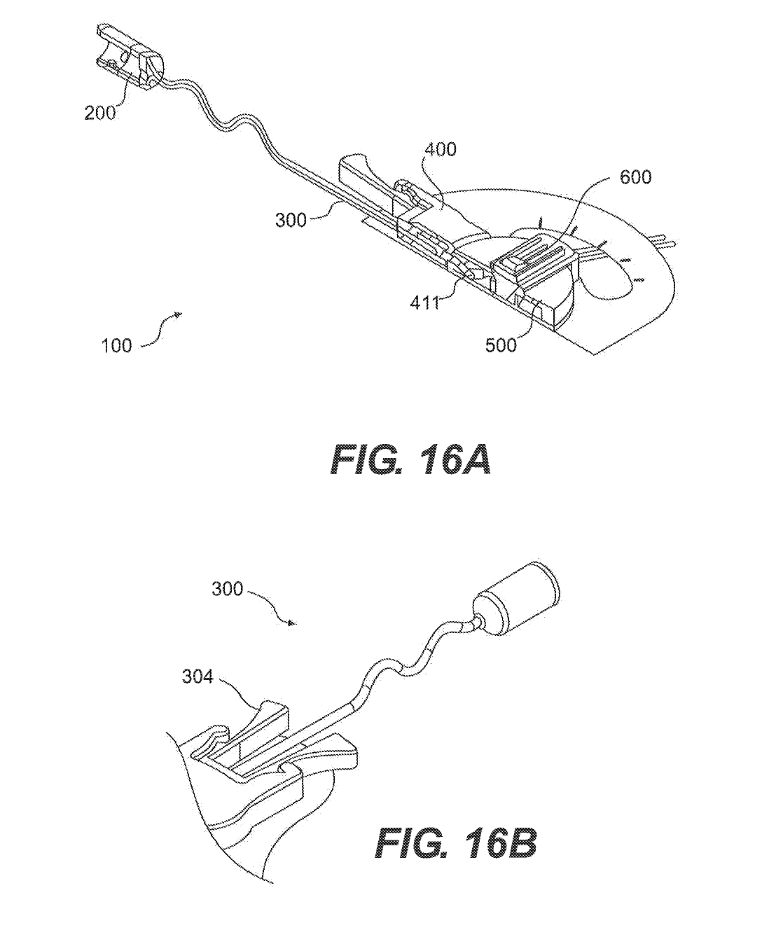

Rotating platform 500 may be configured to rotate around a neutral position, shown in FIGS. 14A-14C and 16A. Rotating platform 500 may rotate any suitable number of degrees in either direction away from the neutral position, for example, 30 degrees from the neutral position on either side. Additionally, cannulae 600, 700 may be configured to allow for insertion into the body of the user at multiple angles 101, as is shown in FIGS. 14B and 14C, to accommodate the preference or needs of the patient. For example, device 100 may be configured to allow a user to change angle 101 once inserted in the body, or to change angle 101 prior to insertion. Additionally, device 100 may include a locking mechanism or fastener to fix the desired angle in place by fixing rotating platform 500 with respect to base platform 400 once the angle is selected and to reduce the shifting of cannula 600, 700 once inserted.

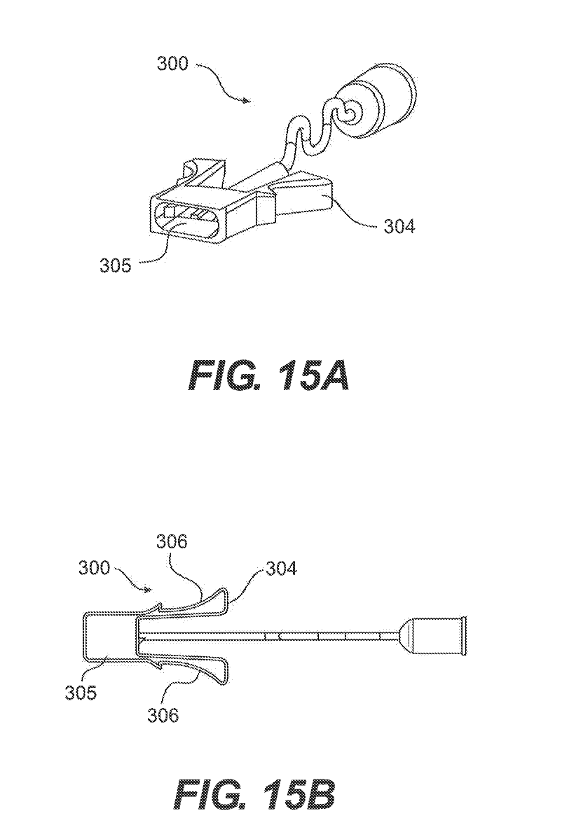

Connector 300 may include a clip 304 comprising a pair of flexible members 306 (FIGS. 15A and 15B) to attach connector 300 to base platform 400 and lock connector 300 in place, as is shown in FIGS. 16A and 16B. Clip 304 may be ergonomically configured for comfort and to facilitate extraction and attachment of connector 300 and base platform 400. For example, clip 304 may lock connector 300 to base platform 400 by pushing connector 300 into a receiving portion of base platform 400. The user may release connector 300 from base platform 400 by squeezing flexible members 306 of clip 304 towards each other.

In some embodiments, connector 300 may include a housing 305 (FIGS. 15A and 15B) containing needles, catheters, or connecting elements that may include sharp or fragile portions. Housing 305 may be configured for safer handling; for example, the shape of housing 305 and connector 300 may allow for sharp or fragile objects to be recessed within housing 305. Such a configuration may decrease the risk of accidental injury of the user and/or the bending or detachment of these portions.

Device 100 may further include flexible tubing 411, shown in FIG. 16A, configured to transport fluids and sensor components from connector 300 and into base platform 400 and into cannulae 600, 700. Tubing 411 may allow rotating platform 500 to freely rotate while decreasing the occurrence of leaks or ‘dead zones’ where fluids may accumulate and/or stagnate. Tubing 411 may be used in conjunction with the various channels of connector 300, base platform 400, rotating platform 500, and cannulae 600, for example, to line the channels or to reinforce the channels at points of intersection.

In the exemplary embodiments, detecting additional patient parameters, e.g., vital signs, including heart rate, blood pressure, blood oxygen, or carbon dioxide levels, may also be desirable for the administration of medicaments. Sensor cannula 700 may include one or more of one type of sensor, or may include different types of sensors for measuring different parameters of the patient. Some embodiments of the present disclosure may have the capability to add or substitute additional sensors for detecting a range of physical characteristics pertinent to the patient or physician. In other embodiments, multiple sensor cannulae 700 may be used. For example, in one embodiment, a thermometer may be placed on the device, e.g., inside of patch 401 or sensor cannula 700, to detect body temperature. In some embodiments, device 100 may be configured to regulate the temperature of the medicament to be delivered. For example, device 100 may include miniature, portable chillers and/or heaters for maintaining the requisite temperatures of certain medicaments.

Some embodiments of the present invention may include a wireless transmitter capable of transmitting the information from the sensors to a pump or medicament source even if a direct connection via the sensor channels has been severed, as will be discussed further below. In some embodiments, base platform 400 may include a display to output information received from sensor cannula 700 to an observer, as will be discussed in further detail below.

The sensor may include any suitable housing containing relevant electronics. Further, the sensor may sense a user's blood glucose by any known sensing technologies, including, but not limited to, technologies employing chemical and/or optical sensing technologies. Such sensors may utilize existing as well as future sensors to incorporate advances in sensing technologies. Any suitable type of sensor can be used for monitoring any body parameter, for example, cholesterol, hormone levels, etc.

In the depicted embodiments, connector 300 of device 100 may be assembled first, by attaching the proximal end of catheter 303 to inlet interface 301 and attaching the distal end of catheter 303 to platform interface 302. Pump outlet 200 may then be attached to inlet interface 301. Patch 401 may be attached to base platform 400, as seen in FIG. 3. Membrane 501 may then be attached to rotating platform 500 or base platform 400. At that point, rotating platform 500 may be attached to base platform 400. Connector 300 may then be attached to base platform 400. Cannulae 600, 700 may be attached to the user, for example, inserted under the skin, with or without the aid of optional cannulae introducers 800, 900, as seen in FIGS. 11A-11D.

Further, any of the channels in pump outlet 200, connector 300, base platform 400, and cannulae 600, 700 may include one or more valves (not shown) in order to prevent backflow of the fluid within the channels. The valves may be active or passive valves. For example, in some embodiments, feedback control may allow for automatic opening or closing of the valve and dispersion of the medicament. Alternatively, the valves may be self-actuating valves configured to open or close based on changes in fluid pressure as fluid is discharged from pump outlet 200.

The method of delivering medicament using the drug delivery device, such as the exemplary infusion sets disclosed herein, may include placing an infusion set on a body part of a user, attaching the infusion set to the user's body, attaching the infusion set to the pump outlets, and commencing medicament delivery from a drug delivering device. The method of delivering medicament using a drug delivery device may further include the step of connecting an infusion set to the drug delivery device. The step of delivering fluid medicament may include delivering fluid medicament at either a controlled and continuous rate or a variable rate for a predetermined or user-defined period of time. Alternatively, the step of delivering fluid medicament may further include delivering fluid medicament at a programmable rate that is regulated, e.g., by the user or by a remote healthcare provider.

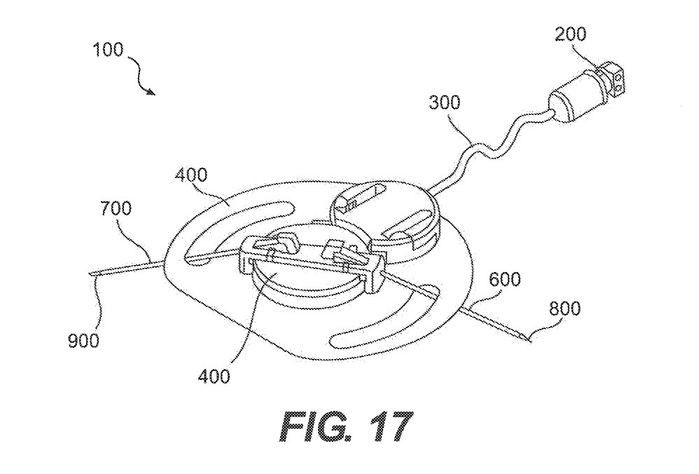

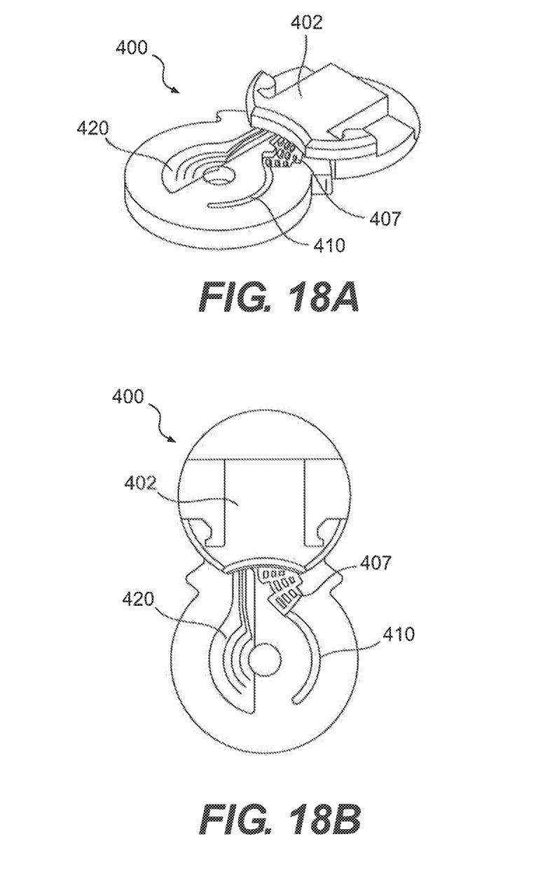

Another embodiment of a device 1000 is depicted in FIGS. 17, 18A, and 18B. Device 1000 may include pump outlet 200, connector 300, base platform 400, rotating platform 500, medicament delivery cannula 600, sensor cannula 700, and optional cannulae introducers 800, 900. Device 1000 may share many of the features of the previous embodiment, except that the two fluid medicaments may be combined prior to injection under the skin. As seen in FIGS. 18A and 18B, device 1000 may further include a mixing chamber 407 configured to combine two or more medicaments from channels 412, 413 from the connector 300. Mixing chamber 407 may mix the fluids using any suitable mixing structure. In the depicted embodiment, mixing chamber 407 uses a maze-like structure to combine the fluids, before leading the mixed fluid into rotating platform 500 in a manner similar to the previous embodiment. Maze-like channels or intersecting channels may be a simple and effective method to mix fluids. Examples of such mixing methods are discussed in J. Melin, G. Gimenez and N. Roxhed, “A fast passive and planar liquid sample micromixer”, Lab Chip, vol. 4, pp. 214-219, 2004, which is incorporated by reference. Embodiments may include other passive ways to mix fluids, e.g. lamination, zigzag channels, three-dimensional serpentine structures, embedded barriers, slanted wells or twisted channels. Examples of such mixing structures are discussed in C. Y. Lee, C. L. Chang, Y. N. Wang, and L. M. Fu, “Microfluidic mixing: a review, Int J. Mol. Sci., vol. 12, pp. 3263-3287, 2011, which is incorporated herein by reference. Still other embodiments may include active methods of mixing fluids. Because the different fluids are mixed into a single fluid prior to injection, cannula 600 may contain only a single channel 601 for delivering the fluid under the skin of the user, as seen in FIG. 17.

Embodiments may include a catheter 303 with any number of channels to deliver any number of medicaments to the user. Medicaments may be mixed after delivery to a user, as in the embodiment of FIGS. 1A and 1B. Alternatively, medicaments may be mixed in device 1000, as is depicted in FIGS. 17, 18A, and 18B, via a mixing chamber 407 included in the infusion set on base platform 400. In another alternative, medicaments may be pre-mixed, for example, in a pump or delivery device external to device 100.

In some embodiments (not shown), cannulae 600, 700 may be adjustable and configured to enter under the skin at a range of angles. The desired angle may be determined according to user comfort, the type of medicament to be delivered, the desired injection depth, or any other suitable factor.

Device 100 may be actuated either manually or through automatic means, for example, through the use of a programmable controller. For example, a user may initiate medicament delivery. Embodiments of the present disclosure may include one or more actuation mechanisms, for example, a button, a switch, a lever, a knob, or a trigger, to manually deliver a precise dose of medicament to a user. Actuation mechanisms may be located on any suitable surface of device 100 and may be configured to control one or more behaviors of device 100, e.g., switch on or off, commence or stop medicament delivery, initiate a measurement, power on and off device 100, or perform any other suitable behavior.

Alternatively, device 100 may be an “intelligent” device that is computer controlled. For example, device 100 may be programmable and may be configured to deliver an appropriate dosage either continuously or at discrete intervals, as desired. Medicament delivery may be triggered by a preprogrammed algorithm within the electronics of device 100, a handheld controller, or a Bluetooth device (not shown). Accordingly, device 100 may be designed to communicate wirelessly with a control device, such as a smart phone, and may be programmed via an application by a healthcare provider or a user. Moreover, medicament delivery may be selectively triggered by a user via the handheld controller or a Bluetooth device. Alternatively, medicament delivery may be triggered by an application programmed to initiate delivery when desired, or by a healthcare provider monitoring the patient through an application and capable of triggering the bolus event remotely.

For example, some embodiments of the present disclosure may use a far-field radio frequency communication system to integrate device 100 with a control unit, for example, a hand-held remote control device. Those of ordinary skill will recognize that any suitable wired or wireless (e.g., infrared, Bluetooth, Wi-Fi, etc.) communication system may be used. Device 100 may further include a digital remote controller that wirelessly communicates with device 100, operating and controlling the delivery of the medicament. Further, the control unit may include a data acquisition system, for example, a program or series of algorithms, configured to store or process data input from the sensor.

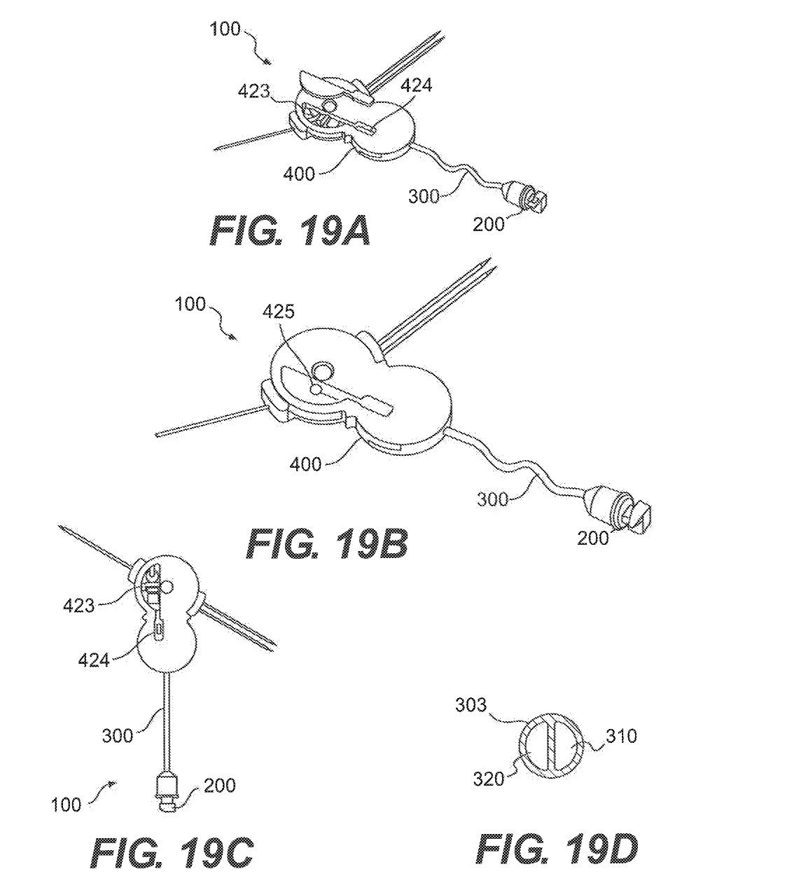

For example, the embodiments depicted in FIGS. 19A-19C include a wireless communication device 423 and an antenna 424. Wireless communication device 423 may transmit information from sensor cannula 700, for example, patient parameters (such as blood glucose levels, heart rate, blood pressure cholesterol levels, temperature etc.), medicament delivery status, or other suitable information to a receiving device. Device 100 may include additional sensors, for example, a temperature sensor 425, to detect and then transmit pertinent information. Further, this data from sensor cannula 700 may communicate with the remote controller to control the timing, rate, amount, or other aspects of medicament delivery, or to provide feedback to the user or a healthcare provider, whether on location or remote. In such embodiments, device 100 may not contain separate channels for sensor components, as is demonstrated in FIG. 19D, because no wires may be needed to relay data from sensor cannula 700 to a proximal region of device 100. FIG. 19D shows catheter 303 including only two fluid channels 310, 320 and omitting sensor component channel 330.

In addition to this function, a complete history of medicament delivery may be stored in device 100 for use by the user or by a healthcare provider for assessment and monitoring of the patient's healthcare. The stored history may be communicated, e.g., wirelessly, to a central database or the healthcare provider for evaluation. Evaluation may occur either with the user directly, for instance the data may be downloaded during a patient visit, or remotely, for instance, transmitted to a database on an ongoing basis.

In addition, device 100 may be operably coupled to a controller with a memory or a processor configured to store and/or process information regarding medicament delivery events and/or patient parameters. The controller may be located on device 100 or may be external to device 100. For example, the controller may be incorporated into the user's mobile device, or may be incorporated in a remote patient monitoring device, e.g., located at a health care facility. Information may be logged and time stamped, allowing the patient or physician to better track and/or analyze drug delivery history and/or user response, and to ultimately improve patient care. This information may be relayed through a wireless connection to a healthcare provider so that the provider may track dosing and/or patient data, such as patient response to the medicament, in real time or from a stored history, from a remote location. In addition, the provider may be able to adjust the dosages and/or medicament type remotely. In some embodiments, the controller may automatically control dosing, e.g., through a timer, or through the use of feedback, or a user or healthcare provider may be able to manually control the timing and dosage. In other embodiments, both may be possible, for example, device 100 may have automatic and manual modes, or device 100 may be automated, but may also have a manual override option.

Further, device 100 and/or an external controller may provide information and/or feedback and/or readings to a user or provider, through, e.g., visual signals on a display or through tactile or auditory signals. Base platform 400 and/or an external pump may include a display screen, e.g., a graphical display screen. The display may be configured to display one or more system parameters (e.g., battery life, error messages), the current time and date, monitored body parameters, date and time of last dose, and any other information that may be communicated to a user of device 100. This information may be conveyed using written words, symbols, pictures, colors, or any other suitable visual means of communication.

Further, in some embodiments, it is contemplated that device 100 may include additional optional features. Such features may include, but are not limited to, circuitry relating to fitness and/or a user's lifestyle. For example, the system may include an integrated pedometer, a global positioning system (GPS), a music player, and so forth. This may be ideal for, for example, diabetic patients who have been prescribed exercise as part of their health regimen. In other embodiments, the system may be configured to integrate with a device, such as a watch, computer, or a smart phone, e.g., through an application or program, to allow a user or a healthcare provider to control the system and/or monitor drug delivery, patient parameters, or patient response, from the external device.

Embodiments of the disclosed device may be powered by a one or more batteries (not shown) located in the housing. The batteries may be any suitable batteries known in the art. In some embodiments, the batteries may be single-use batteries, or in other embodiments, the batteries may be batteries that may be selectively rechargeable. In such instances, the batteries may be removed from device 100 and placed into a suitable recharging apparatus until power is fully restored. In even further embodiments, the batteries may be configured to be recharged without requiring removal from within device 100, for example, recharged wirelessly.

While the present invention is described herein with reference to illustrative embodiments for particular applications, it should be understood that the invention is not limited thereto. Those having ordinary skill in the art and access to the teachings provided herein will recognize additional modifications, applications, embodiments, and substitution of equivalents all fall within the scope of the invention. Accordingly, the invention is not to be considered as limited by the foregoing description.

What is claimed is:

1. A medical device for delivering medicament to a body of a user, the medical device comprising:
a connector for channeling a fluid from a fluid source;
a device platform configured to receive the fluid from the fluid source via the connector, the device platform being configured to contact the body of the user;
one or more fluid cannulae coupled to and extending from the device platform, the one or more fluid cannulae being configured to receive the fluid from the device platform and configured for insertion into the body of the user;
one or more channels for delivering the fluid to the body of the user, the one or more channels being configured to channel the fluid through the connector, into the device platform, and into the one or more fluid cannulae; and
one or more sensors coupled to the device platform and configured for monitoring a parameter of the body.

2. The medical device of claim 1, wherein the one or more sensors are included in a sensor cannula coupled to the device platform and the sensor cannula is configured for insertion into the body of the user.

3. The medical device of claim 1, wherein the one or more sensors are wirelessly coupled to a controller located external to the medical device.

4. The medical device of claim 1, wherein the one or more sensors include a continuous blood glucose monitor and the fluid includes insulin.

5. The medical device of claim 1, wherein the one or more channels include at least one channel configured to carry one or more sensor wires from the one or more sensors, through the device platform, through the connector, and to a proximal region of the medical device.

6. The medical device of claim 1, wherein data received from the one or more sensors are used to control the delivery of fluid to the user.

7. A medical device for delivering medicament to a body of a user, the medical device comprising:
a connector for channeling at least a first fluid and a second fluid from one or more fluid sources;
a device platform configured to receive the first fluid and the second fluid from the connector, the device platform being configured to contact the body of the user;
one or more cannulae coupled to the device platform, at least one of the one or more cannulae being configured to receive the first fluid and the second fluid from the device platform and configured for insertion into the body of the user; and
one or more channels for delivering the first fluid and the second fluid to the body of the user, the one or more channels being configured to channel the first fluid and the second fluid through the connector, into the device platform, and into the one or more cannulae.

8. The medical device of claim 7, wherein the first fluid and the second fluid are different types of medicaments.

9. The medical device of claim 7, wherein the one or more channels includes a first channel for carrying the first fluid and a second channel for carrying the second fluid, and wherein the first fluid and the second fluid are delivered separately to the body of the user.

10. The medical device of claim 7, wherein the one or more channels includes a first channel for carrying the first fluid and a second channel for carrying the second fluid, and wherein the first channel and the second channel join together inside of the medical device to form a combined channel, such that the first fluid and the second fluid are carried together in the combined channel.

11. The medical device of claim 10 wherein the device platform further comprises a mixing chamber configured to mix the first fluid and the second fluid; wherein the first channel and the second channel are configured to channel the first fluid and the second fluid into the mixing chamber; and the combined channel is configured to channel the first fluid and the second fluid into the one or more cannulae for delivery of the first fluid and the second fluid to the body of the user.

12. A medical device for delivering medicament to a body of a user, the medical device comprising:
a connector for channeling a fluid from a fluid source;
a device platform configured to receive the fluid from the connector, the device platform being configured to attach to the body of the user;
one or more cannulae coupled to the device platform, at least one of the one or more cannulae being configured to receive the fluid from the device platform and configured for insertion into the user; and
one or more channels for delivering the fluid to the body of the user, the one or more channels being configured to channel the fluid through the connector, into the device platform, and into at least one of the one or more cannulae;
wherein the device platform is configured to rotate such that the one or more cannulae is configured to be withdrawn from the body of the user, such that the one or more cannulae rotates with the device platform, and such that the one or more cannulae is configured to be re-inserted into the body of the user in a different location without detaching the device platform from the body of the user.

13. The medical device of claim 12, wherein the device platform is configured to allow a user to increase or decrease the angle of insertion of the one or more cannulae into the body of the user.

14. The medical device of claim 12, wherein the device platform includes gaps through which the one or more cannulae may extend to contact the body of the user.

15. The medical device of claim 12, wherein the one or more channels extend in an arc around a portion of the device platform such that the length of the one or more channels in the device platform corresponds to the full range of rotation at which the one or ore cannulae coupled to the device platform can be rotated.

16. The medical device of claim 12 further comprising:
a locking mechanism configured to prevent further rotation of the device platform once a desired rotational position has been achieved.

17. The medical device of claim 12, further comprising a sensor operably coupled to the device platform and configured to measure a parameter of the user.

18. The medical device of claim 17, wherein the sensor is disposable.

19. The medical device of claim 17, wherein the sensor is configured to attach to the body of the user, the device platform is configured to rotate, and the sensor is configured to be detached from the body of the user, rotate with the device platform to a new location, and re-attached to the body of the user in the new location, without removing the device platform from the body of the user.

20. The medical device of claim 12, wherein the one or more cannulae includes at least one fluid cannulae configured to receive one or more fluids and at least one sensor cannulae configured to contain one or more sensors.

21. The medical device of claim 20, wherein at least one of the one or more fluid cannulae and the one or more sensor cannulae is disposable and configured to be withdrawn from the body of the user and rotated with the device platform, and at least one of the fluid cannulae and the sensor cannulae is configured to be replaced with a new fluid cannulae or a new sensor cannulae, and re-inserted into the body of the user.

22. The medical device of claim 12, wherein the medical device is configured to deliver a first fluid and a second fluid to the body of the user.

23. The medical device of claim 22, wherein the one or more cannulae includes a first fluid cannulae configured to deliver the first fluid and the second fluid to the body of the user.

24. The medical device of claim 22, wherein the one or more cannulae includes a sensor cannulae, a first fluid cannulae, and a second fluid cannulae, wherein the first fluid is delivered to the body of the user through the first fluid cannulae, and the second fluid is delivered to the body of the user through the second fluid cannulae.

25. The medical device of claim 24, wherein the sensor cannulae includes a continuous glucose monitor and the first fluid is insulin.
